(19) United States
(12) Patent Application Publication
(10) Pub. No.: US 2022/0071558 A1
(43) Pub. Date: Mar. 10, 2022

(54) SYSTEM, DEVICE, AND METHOD FOR WIRELESS HEALTH MONITORING

(71) Applicant: LEDO Network, Inc., Cupertino, CA (US)

(72) Inventors: Bryan He Huang, Cupertino, CA (US); Hong Wang, Cupertino, CA (US)

(21) Appl. No.: 17/208,813

(22) Filed: Mar. 22, 2021

Related U.S. Application Data
(63) Continuation-in-part of application No. 17/014,657, filed on Sep. 8, 2020.

(51) Int. Cl. A61B 5/00; A61B 5/01; A61B 5/08

(52) U.S. Cl. CPC A61B 5/4818; A61B 5/4812; A61B 5/01; A61B 5/0816; A61B 5/0077; A61B 5/7275; A61B 2503/12; A61B 5/6808; A61B 5/0002; A61B 2560/0252; A61B 2503/04; A61B 2503/06; A61B 5/746

(57) ABSTRACT

A method for monitoring health of a person by a health monitoring system includes: measuring movement data of a person using a wearable health monitoring device, determined whether the person is in a deep sleep mode based on the movement data, automatically measuring a respiratory rate of the person that is in a deep sleep mode, determining that the person is in one of a plurality of respiration zones, measuring at least one of a body temperature, an ambient temperature, a sleeping position, a smoke or carbon monoxide level, a video, and images of the person, and automatically producing an alarm signal if a predetermined criterion is met based on the respiration zone and at least one of the body temperature, the ambient temperature, the smoke level, the carbon monoxide level, a sleeping position, or the images of the person.

### BACKGROUND OF THE INVENTION

The present invention relates generally to the field of healthcare technologies, and in particular, to a wearable device and a related system for monitoring health of a person, such as an infant.

Every year tens of thousands of babies die from Sudden Infant Death Syndrome (“SIDS”) around the world. While specific causes of SIDS might be difficult to determine, researchers have discovered a combination of physical and sleep environmental factors can make an infant more vulnerable to SIDS. Physical factors associated with SIDS can include respiratory issue: for example, many infants who died of SIDS had recently had a cold, which might contribute to breathing problems; and low birth weight: Premature birth increases the likelihood that a baby's brain hasn't matured completely, so he or she has less control over such automatic processes as breathing and heart rate. Sleep environmental factors can include: sleeping on the stomach or side: Babies placed in these positions to sleep might have more difficulty breathing than those placed on their backs; overheating: Being too warm while sleeping can increase a baby's risk of SIDS; and secondhand smoke: Babies who live with smokers have a higher risk of SIDS; and sharing a bed with parents, siblings or pets which increases risks of SIDS.

Nowadays, many parents use video cameras or audio monitors to watch or listen to their babies, for motion or audio detection. These systems, however, do not provide a parent with enough information to intervene before a serious health issue happens to the baby.

There is therefore a need for an improved health monitoring system to address the described problems above.

### SUMMARY OF THE INVENTION

In one general aspect, the present invention relates to a method for monitoring health of a person by a health monitoring system. The method includes: measuring movement data of a person using a wearable health monitoring device, determining whether the person is in a deep sleep mode based on the movement data, automatically measuring a respiratory rate of the person that is in a deep sleep mode, determining that the person is in one of a plurality of respiration zones, measuring at least one of a body temperature, an ambient temperature, a sleeping position, a smoke or carbon monoxide level, a video, and the images of the person, and automatically producing an alarm signal if a predetermined criterion is met based on the respiration zone and at least one of the body temperature, the ambient temperature, the smoke level, the carbon monoxide level, the video, and the images of the person.

Implementations of the system may include one or more of the following. The plurality of respiration zones can include a normal zone bounded by a lower normal threshold and an upper normal threshold, wherein the plurality of respiration zones can include a low-respiration risk zone defined by a lower critical threshold below the lower normal threshold, wherein the alarm can be automatically produced if the respiratory rate is below the lower critical threshold. The plurality of respiration zones can further include a lower intermediate zone between the low-respiration risk zone and the normal zone, wherein the alarm sensitivity level is set to the highest level if the person is determined in the lower intermediate zone. The alarm can be produced if the person is determined to be sleeping on stomach or a side based on the sleeping position measurement from the wearable health monitoring device or the video monitor. The plurality of respiration zones can further include an intermittent zone and a high-respiration risk zone having respiratory rates above the upper normal threshold, wherein the person can be determined to be in the intermittent zone with no alarm being produced if the respiratory rate is above the upper normal threshold but for less than a predetermined period of time, and the alarm sensitivity level is set to the highest level if the person is determined in the intermittent zone. The person can be determined to be in the high-respiration risk zone and the alarm is produced if the respiratory rate is above the upper normal threshold but for longer than the predetermined period of time. The alarm can be produced if the body temperature of the person is out of a pre-specified range. The method can further include automatically measuring an ambient temperature or ambient humidity, wherein the alarm signal is produced if the ambient temperature or the ambient humidity is determined to be outside of a safe range. The method can further include automatically adjusting the levels of ambient temperature or the ambient humidity if measured ambient temperature or measured ambient humidity is respectively out of a pre-specified range. The alarm signal is produced if the smoke level or the carbon monoxide level is outside a predetermined safe range. The method can further include measuring the person's body weight and automatically updating the growth chart of a person. The person can be an infant, baby, toddler, teenager or adult. The wearable health monitoring device can be attached to or removably disposed in a wearable article worn adjacent to the baby's abdomen.

In another general aspect, the present invention relates to a health monitoring system that includes: a wearable health monitoring device that includes one or more movement sensors configured to produce movement data of a person that wears the wearable health monitoring device; and one or more computer processors that can determine whether the person is in a deep sleep mode based on the movement data. When the person is determined to be in the deep sleep mode, the wearable health monitoring device is configured to measure a respiratory rate of the person. The one or more computer processors can determine that the person is in one of a plurality of respiration zones. An alarm signal is produced if a predetermined criterion is met based on the respiration zone and at least one of a body temperature, an ambient temperature, a sleeping position, a smoke or carbon monoxide level, a video, and images of the person.

In another general aspect, the present invention relates to a method for monitoring health of a person by a health monitoring system, comprising: determining, based on movement data, if a person wearing a wearable health monitoring device is sleeping or not, wherein the movement data is produced by one or more movement sensors in the wearable health monitoring device; when the person is determined to be sleeping, measuring sleep movements of the person to determine whether the person is in a deep sleep mode; measuring one or more bio-vital signals of the person and at least one of a sleeping position/posture of the person or one or more environmental parameters when the person is determined to be in the deep sleep mode, wherein the one or more bio-vital signals are produced by one or more bio-vital signal detectors in the wearable health monitoring device; and automatically producing an alarm signal if a predetermined criterion is met based on the one or more bio-vital signals and at least one of a posture of the person or the one or more environmental parameters.

Implementations of the system may include one or more of the following. The one or more bio-vital signals can include a respiratory rate. The method can further include: automatically determining by a health monitoring system whether the person has a slow respiratory rate or a fast respiratory rate by comparing the respiratory rate to a predetermined respiration threshold, wherein the health monitoring system includes the wearable health monitoring device. The method of claim can further include when the person is determined to have a slow respiration, including temporal shortness of breath, automatically producing the alarm signal if the respiratory rate is below a critical respiration threshold. The method can further include: when the person is determined to have a slow respiration, automatically setting the alarm sensitivity level to the highest level if the person is determined in the lower intermediate zone. The method can further include: when the person is determined to have a fast respiration, automatically determining whether respiration behaviors are within an intermittent zone based on an absolute value of the respiratory rate and a period of time within which the respiratory rate is above the predetermined respiration threshold, and setting the alarm sensitivity level to the highest level if the person is determined in the intermittent zone; and automatically producing an alarm signal if the respiration behaviors are outside of the intermittent zone. The respiration behaviors are outside of the intermittent zone when the respiration behaviors are above a safe respiration threshold, or the person is determined to have a fast respiration for an extended period long than a threshold period, or a combination thereof. The method can further include: when the person is determined to have a fast respiration, measuring one or more environmental parameters; automatically determining by a health monitoring system whether the one or more environmental parameters are within respective desirable ranges, wherein the health monitoring system includes the wearable health monitoring device, wherein the alarm signal is produced if the one or more environmental parameters are determined to be outside of respective desirable ranges. The one or more environmental parameters can include ambient temperature or humidity. The person can be an infant, baby, toddler, teenage, or adult. The wearable health monitoring device can be attached to or removably disposed in a wearable article worn by and in contact with the baby's abdomen. The one or more movement sensors in the wearable health monitoring device can include one or more of an accelerator, a magnetic detector, a digital compass, a gyroscope, a pressure sensor, an inertia module, or a piezoelectric sensor. The one or more bio-vital signal detectors in the wearable health monitoring device can include one or more of a body temperature sensor, a respiratory sensor, a blood pulse sensor, a blood oxygen sensor, a humidity sensor, a noise sensor, or one or more electric signal sensors.

In another general aspect, the present invention relates to a health monitoring system that includes a wearable health monitoring device comprising one or more movement sensors that can produce movement data of a person that wears the wearable health monitoring device, wherein the wearable health monitoring device includes one or more bio-vital signal detectors that can produce one or more bio-vital signals; and one or more computer processor that can determine, if the person is sleeping or not based on movement data, wherein the one or more movement sensors can measure sleep movements of the person when the person is determined to be sleeping, wherein the one or more computer processors can determine whether the person is in a deep sleep mode, wherein when the person is determined to be in the deep sleep mode, the one or more computer processors can produce an alarm signal if a predetermined criterion is met based on the one or more bio-vital signals and at least one of a posture of the person.

Implementations of the system may include one or more of the following. The one or more bio-vital signals include a respiratory rate, wherein the one or more computer processors can automatically determine whether the person has a slow respiration or a fast respiration by comparing the respiratory rate to a predetermined respiration threshold. When the person is determined to have a slow respiration, the one or more computer processors can produce the alarm signal if the respiratory rate is below a critical respiration threshold. The wearable health monitoring device includes an accelerometer, wherein when the person is determined to have a slow respiration, the one or more computer processors can automatically adjust the alarm sensitivity level to high if the person is determined in the lower intermediate zone. When the person is determined to have a fast respiration, the one or more computer processors can automatically determine whether respiration behaviors are within an intermittent zone based on an absolute value of the respiratory rate and a period of time within which the respiratory rate is above the predetermined respiration threshold, wherein the one or more computer processors can automatically produce an alarm signal if the respiration behaviors are outside of the intermittent zone, and automatically adjust the alarm sensitivity level to the highest level if the person is determined in the intermittent zone. The respiration behaviors are outside of the intermittent zone when the respiration behaviors are above a safe respiration threshold, or the person is determined to have a fast respiration for an extended period long than a threshold period, or a combination thereof. The health monitoring system can further include one or more environmental sensors configured to produce one or more environmental parameters, when the person is determined to be in the deep sleep mode, the one or more computer processors can further produce an alarm signal if a predetermined criterion is met based on the one or more bio-vital signals and at least one of a posture of the person or one or more environmental parameters. When the person is determined to have a fast respiration, one or more environmental sensors can measure one or more environmental parameters, wherein the one or more computer processors can automatically determine whether the one or more environmental parameters are within respective desirable ranges, wherein the one or more computer processors can automatically produce the alarm signal if the one or more environmental parameters are determined to be outside of respective desirable ranges, wherein the one or more environmental parameters include ambient temperature, humidity, electro-magnetic field strength level, or noise. The person can be an infant, baby, or toddler, wherein the wearable health monitoring device is attached to or removably disposed in a wearable article worn by and in contact with the baby's abdomen. The one or more movement sensors include one or more of an accelerator, a magnetic detector, a digital compass, a gyroscope, a pressure sensor, an inertia module, or a piezoelectric sensor. The one or more bio-vital signal detectors include one or more of a body temperature sensor, a humidity sensor, a respiratory sensor, a blood pulse sensor, a blood oxygen sensor, a noise sensor, or one or more electric signal sensors.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims.

### BRIEF DESCRIPTION OF THE DRAWINGS

FIG. 1 is a system diagram for monitoring the health of a person in accordance with some embodiments of the present invention.

FIGS. 2A-2E illustrate a wearable health monitoring device in accordance with some embodiments of the present invention. FIG. 2A: a front perspective view of the wearable health monitoring device clipped onto a wearable article; FIG. 2B: an exploded perspective view of the wearable health monitoring device; FIG. 2C: a front view of the wearable health monitoring device; FIG. 2D: a rear perspective view of the wearable health monitoring device; FIG. 2E: a left perspective view of the wearable health monitoring device.

FIG. 3 illustrates a removable wearable health monitoring device that can be disposed within a swaddle blanket in accordance with some embodiments of the present invention.

FIG. 4 is an exemplary block diagram of a wearable health monitoring device in accordance with some embodiments of the present invention.

FIG. 4A shows exemplary movement sensors suitable for the disclosed wearable health monitoring device.

FIG. 4B shows exemplary bio-vital signal detectors suitable for the disclosed wearable health monitoring device.

FIG. 4C shows exemplary environmental sensors suitable for the disclosed wearable health monitoring device.

FIG. 5A illustrates an exemplified receiving station in accordance with some embodiments of the present invention.

FIG. 5B depicts an exemplified circuit board and display.

FIGS. 6A-6B depict exemplified mobile device displaying interfaces.

FIG. 7 shows a system diagram of the health monitoring system in accordance with some embodiments of the present invention.

FIG. 8 is a flowchart in accordance with some embodiments of the present invention.

FIG. 9A shows exemplary movement patterns that can be recognized in the disclosed system.

FIG. 9B shows exemplary sleeping parameters that can be calculated by the disclosed system.

FIG. 9C shows exemplary environmental parameters that can be calculated by the disclosed system.

FIG. 10 illustrates a portion of a health monitoring system including several monitoring devices and a mobile phone in accordance with some embodiments of the present invention.

FIG. 11 shows another system diagram of the health monitoring system in accordance with some embodiments of the present invention.

FIG. 12 is another flowchart in accordance with some embodiments of the present invention.

FIG. 13 shows respiration zones that require different monitoring and measurement follow-ups and different alert actions in the operations of the health monitoring system in accordance with some embodiments of the present invention.

### DETAILED DESCRIPTION OF THE INVENTION

The presently disclosed system attempts to address the inadequacy in conventional baby monitoring systems. The disclosed system monitors the health of a person via a wearable health monitoring system, which monitors baby's bio-vital signals such as respiratory rate and body temperature, movement parameters as sleeping position, and ambient conditions, and the smoke/carbon monoxide level, the video, and the images of the person. An alert is produced when a criterion for an abnormally is identified. The disclosed system can be used for babies including newborns, infants, and toddlers, as well as people at older ages, such as teenager, seniors, or patients who are prone to respiratory disruptions during sleep.

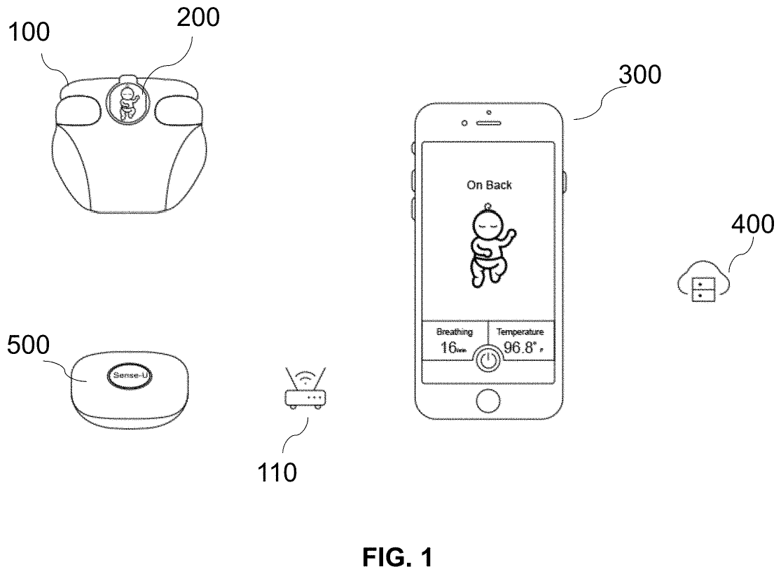

In some embodiments, referring to FIG. 1, a wearable health monitoring device 200 is clipped onto a wearable article 100. The wearable health monitoring device 200 can be in communication with a receiving station 500. The receiving station 500 can be in communication with an internet gateway 110 (e.g., a cable modem, a Wi-Fi router, a DSL modem, etc.). The internet gateway 110 is shown in communication with a computing device such as a mobile device 300. The mobile device 300 can display data that was originally gathered by the wearable health monitoring device 200. Alternatively, the wearable health monitoring device 200 can be in communication with a mobile device 300 directly or via the internet gateway 110 with a receiving station. The internet gateway 110 or the mobile device 300 can be in communication with a cloud server 400. When an abnormal health condition occurs, a parent receives an alert notification, to alert them to immediately check on the status of a baby or infant. The mobile device 300 can display health data collected by the wearable health monitoring device 200.

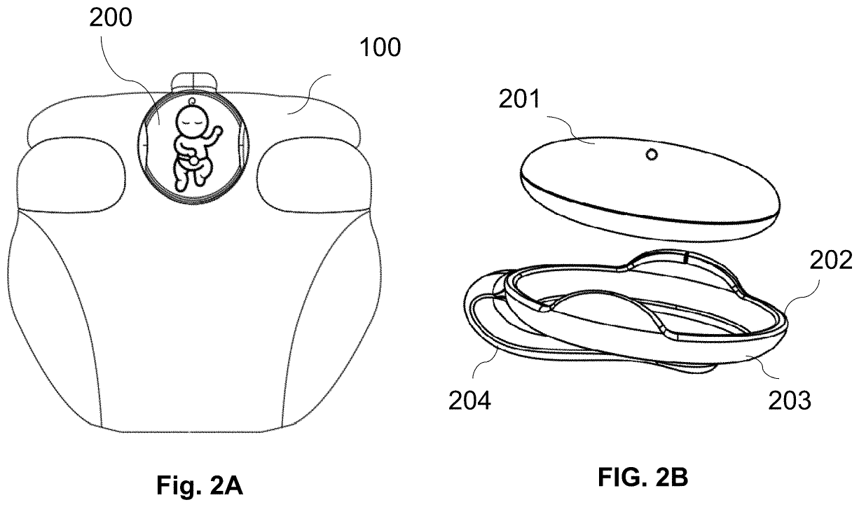

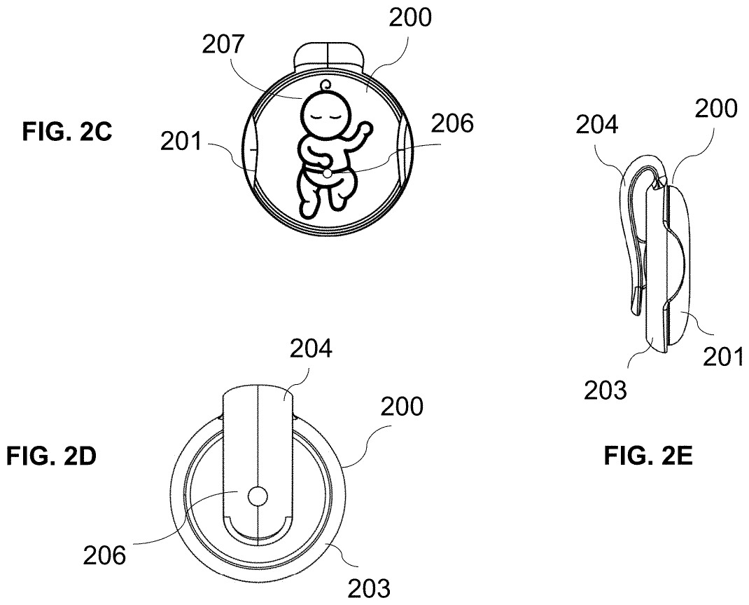

FIG. 2A illustrates an exemplified wearable health monitoring device 200 attached onto or inserted into a wearable article 100 (e.g., a diaper). As shown in FIG. 2B, the exemplified wearable health monitoring device 200 can include a sensor device 201 and a unique securing mechanism 202 including a clip-shaped base 203 and a clip 204. The clip-shaped base 202 is clipped onto the wearable article 100. After pushing the sensor device 201 into the clip-shaped base 202, clipping the wearable health monitoring device 200 onto the wearable article 100, it secures the sensor device 201 in place, creating a tight fastener, which will provide sufficient contact with an infant's abdomen as to detect breathing movement, sleeping position.

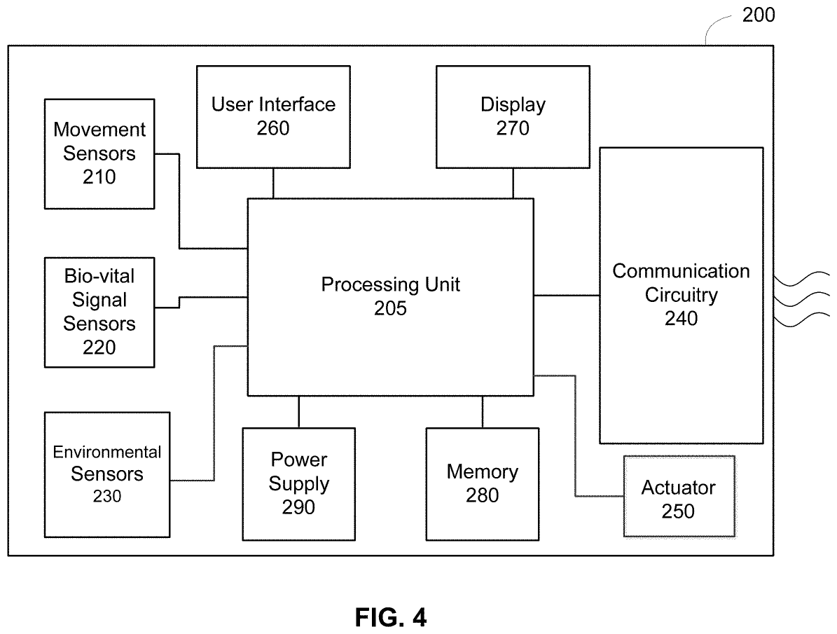

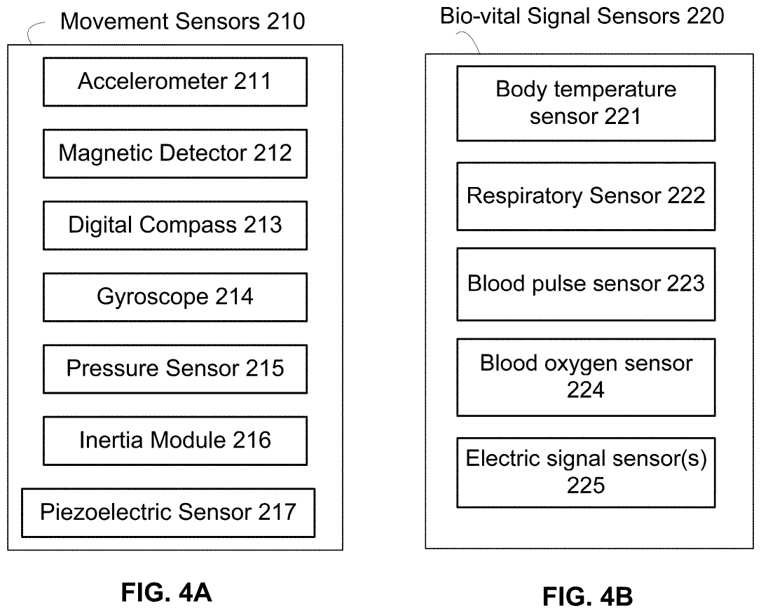

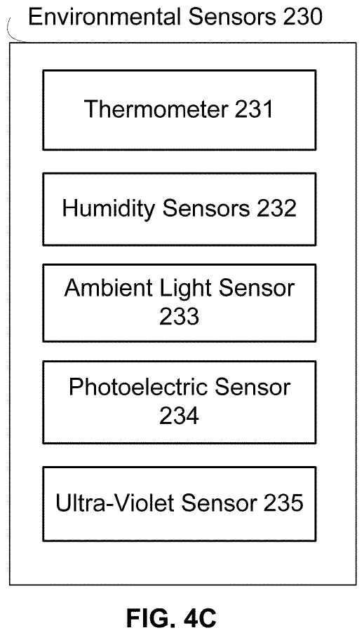

FIGS. 2C, 2D and 2E respectively show the front, the back, and the side views of the wearable health monitoring device 200. The front case of the wearable health monitoring device can have an infant silkscreen 207 illustrating an infant sleeping on his back. It is used to demonstrate the orientation of the wearable health monitoring device together with the recommended sleeping position for an infant. As shown in FIG. 2C, the sensor device 201 can have an infant silkscreen 207 illustrating an infant sleeping on his back, and a temperature contact 206 on one side of the sensor device 201 to measure ambient temperature around the body or directly measure infant's body temperature. To measure body temperature, the device can be worn upside down to touch wearer's skin. As described in detail below, the wearable health monitoring device 200 can include electronics to enable the detection of the movement reading from infant's abdomen. The electrical signals can then be processed by a computer processor (e.g., processing unit 205 in FIG. 4) to generate a respiratory rate value and a sleeping position value as well as other related health data.

The sensor device 201 can include on its side a small temperature contact 206. It can be in the form of a small round hole to measure the ambient temperature and humidity level toward the air, and also to use as a user interface with LED lights showing the health information of the infant, or in the form of a metal contact tip to measure body/skin temperature, with the wearable health monitoring device 200 flipped when clipping onto a wearable article. In some embodiments, the temperature metal contact tip 206 can be located at the back of the clip 204, with the clip-shaped base 203 and the sensor device 201 sharing the same back cover, therefore the wearable health monitoring device 200 can measure body/skin temperature with the silkscreen 207 toward the air. In some embodiments, the sensor device 200 can only measure body temperature while the receiving station 500 measures the ambient temperature.

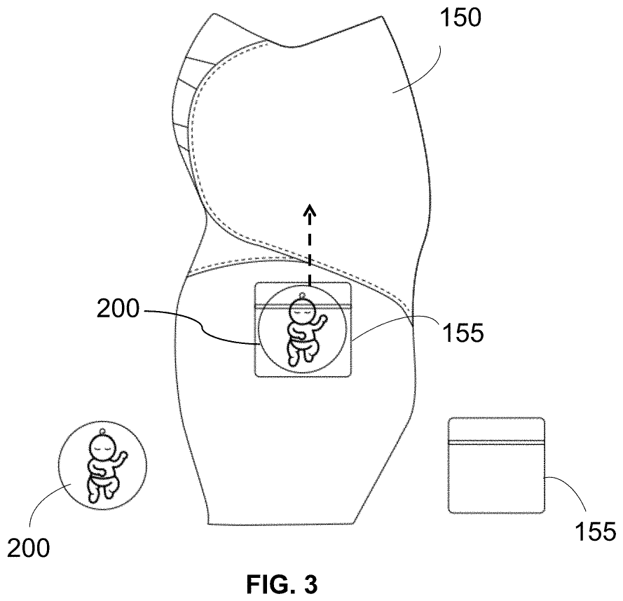

The wearable article 100 can be in the form of a diaper, swaddle blanket, an infant sleeping bag, a wrist band, an elbow or knee pad, a finger ring, or other articles that can be worn by a person. In some embodiments, referring to FIG. 3, a wearable health monitoring device 200 attached onto the wearable article 150 that is in the form of a swaddle blanket that can be wrapped around a person's body. The wearable article 150 can also include a pouch 155 configured to receive the sensor device 201. The pouch 155 can include a zipper that allows the pouch to securely open and close. Additionally, the pouch can include buttons, Velcro, snaps, or other apparatus or useful combination of apparatuses to close the pouch. The sensor device 201, which is receivable into the pouch, can include an outer layer of Velcro or other alignment feature. In particular, the Velcro or alignment feature can be attached to the back of the sensor device 201, which allows the sensor device 201 to be securely fastened to outer strap of the wearable article 150 such that the sensor device 201 is secure and in close contact with the infant's body.

Other details about the above described wearable health monitoring device are disclosed in the commonly assigned U.S. patent application Ser. No. 29/707,085 entitled “Wearable Health Monitor”, filed Sep. 26, 2019, the content of which is incorporated herein by reference.

Referring to FIG. 4, a wearable health monitoring device 200 can be worn on the body of a person such as a baby or an adult for the purpose of health monitoring. The wearable health monitoring device 200 can include movement sensors 210, bio-vital signal sensors 220, and environmental sensors 230, respectively for detecting the activities and the bio-vital signals of the wearing person, and the environment conditions. A processing unit 205 processes the signals detected by movement sensors 210, bio-vital signal sensors 220, and environmental sensors 230 to produce movement data, bio-vital signal data, and environmental data. The wearable health monitoring device 200 can also include one or more actuators 250, a user interface 260, a display 270, a memory 280, and a power supply 290.

FIG. 4A shows examples of the movement sensor 210, which can include one or more of accelerometers 211, magnetic detectors 212, digital compasses 213, gyroscopes 214, pressure sensor 215, inertial module 216, piezoelectric detector 217 and/or other unlisted movement sensors to sense, capture, calculate and record movement data of the object. Different combinations of these movement sensors may be incorporated in the wearable health monitoring devices 200. Even more, all types of detectors, whether listed or unlisted here, that generate data which is representative of the movements of the object, are intended to fall within the scope of the present inventions. The movement sensor 210 can produce signals that represent sleep positions and other movements.

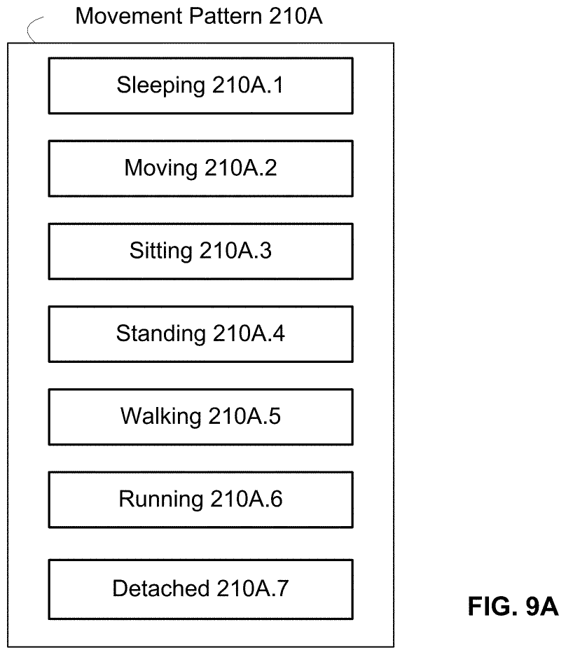

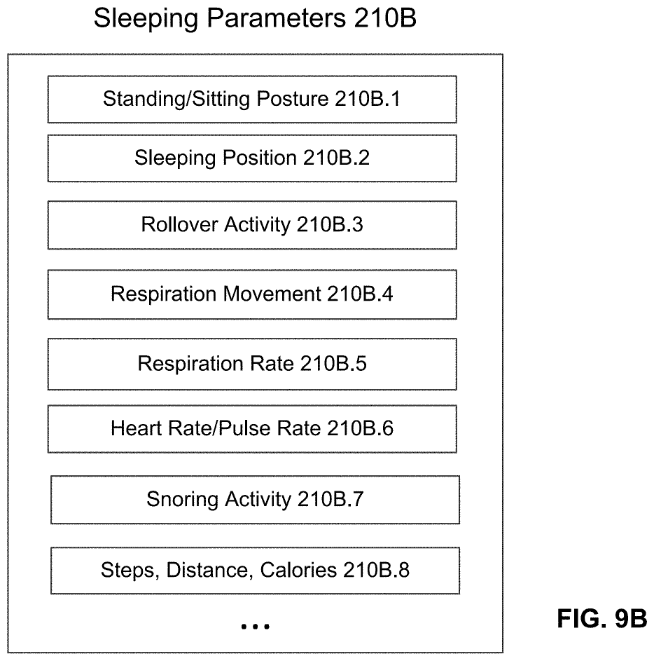

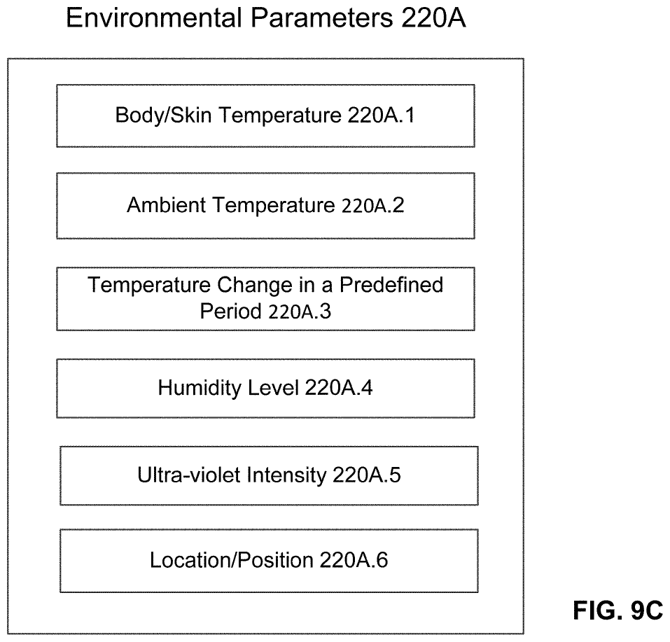

FIG. 4B shows examples of the bio-vital sensors 220, which can include one or more of a body temperature 221, a respiratory sensor 222, a blood pulse sensor 223, a blood oxygen sensor 224, a humidity sensor, a noise sensor, an electro-magnetic field level sensor, and one or more electric signal sensors 225 (such as ECG or EKG). In some implementations, the respiratory sensor 222 can share a same acceleration sensor as the accelerometers 211. The blood pulse sensor 223 can be implemented as a pressure sensor or sometimes using an acceleration sensor. In the latter implementation, in some implementations, the blood pulse sensor 223 can share a same acceleration sensor as the accelerometers 211. The bio-vital sensors 220 can provide sleep data comprising sleep parameters (210B, FIG. 9B).

FIG. 4C shows examples of the environmental sensor 230, which may include one or more of a thermometer 231 for measuring ambient temperature, one or more humidity sensors 232 that can measure ambient humidity and the humidity in a wearable article such as a diaper, an ambient light sensor 233, a photoelectric sensor 234, and a ultra-violet sensor 235. The environmental sensors 230 may also employ other detectors to provide environmental data which is representative of environmental condition of the wearer. The environmental sensors 230 can provide environmental data comprising environmental parameters (220A, FIG. 9C).

The processing unit 205 processes the captured movement data, the bio-vital signal data, and environmental data. The wearable health monitoring device 200 can also include one or more actuators 250. Exemplified functions of actuators can include emitting a sound or a light, producing vibrations, producing heat, ejecting a fluid (such as chemical or medicine), etc. The actuator 250 can be used to alert a parent when a negative health trend is detected. Also, the actuator 250 can be used to stimulate breathing when a negative trend is detected.

In some embodiments, the wearable health monitoring device 200 can include an accelerometer 211 in the movement sensor 210, a body temperature sensor 221 and a respiratory sensor 222 in the bio-vital sensors 220, a humidity sensor 232 in environmental sensors 230, an audio noise sensor, an electro-magnetic wave strength sensor, and a wireless transceiver in a communication circuitry 240. The wearable health monitoring device 200 can be placed on the subject's abdomen to detect respiratory rate and sleeping positions. The accelerometer 211 and the respiratory sensor 222 can share a dual acceleration sensor for capture sleeping position as part of the movement data and respiratory signal as part of bio-vital signal data. The body temperature sensor 221 and the humidity sensor 232 provide the temperature reading and humidity level reading respectively. It is believed that babies are most at risk for SIDS when they sleep on their stomach. Accordingly, the accelerometer 211 and the respiratory sensor 222 can produce data that indicate whether the infant is sleeping on stomach or sleep on their back as well as the infant's breathing status. In determining the infant's breathing or sleeping position, the wearable health monitoring device 200 can conduct readings for a particular amount of time to avoid false alarms.

The processing unit 205 can process breathing movement data on-site. Specifically, the processing unit can process and filter the raw movement data and relaying that data to a mobile device 300 or other types of wireless transceivers. For example, the processing unit can convert the raw data into a format that can be broadcast over a particular wireless connection (e.g., Bluetooth, Zigbee, etc.). One will understand, however, that various transmission formats are known in the art and a combination of known transmission formats can be used and remain within the scope of the present invention.

Additionally, the processing unit 205 can receive raw data signal from the movement sensors 210, the bio-vital signal sensors 220, and the environmental sensors 230, further filter out unwanted noise from movement and external sources. As the processing unit processes and filters the received raw data the processing unit can determine at least a respiratory rate reading and a sleeping position reading.

In addition to the processing unit 205, the wearable health monitoring device 200 can include a power supply 290, such as a battery, that can power the wearable health monitoring device 200. The battery can be removable or rechargeable. The wearable health monitoring device 200 can also include a visual indicator that indicates when the battery is low on power and need replacing.

Once the processing unit 205 receives the raw movement data, the processing unit 205 processes the raw data and calculates the respiration movement 210B.4, respiratory rate 210B.5 and sleeping position 210B.2 (shown in FIG. 9B), stores in the flash memory 280, or other memory device. The processing unit 205 can then send the processed data to the communication circuitry 240 (shown in FIG. 4) that is also located within the wearable health monitoring device.

The communication circuitry 240 and the processing unit 205 can be located on a print circuit board. In some cases, processing the data at the processing unit 205 before transmitting the data with the communication circuitry 240 can result in significant power savings, as compared to transmitting the raw data. Additionally, processing the data with the processing unit 205 before transmitting the data can improve the data integrity and lower the error rate associated with the data.

The communication circuitry 240 can employ different data transfer methods to build the communication link among the wearable health monitoring device 200, the receiving station 500, and the mobile device 300. Communication links can also include, but not limited to, electronic data link, fire wire, a network cable connection, a serial connection, a parallel connection, USB, or wireless data connection, including but not limited to Bluetooth, Bluetooth Low Energy, WLAN, Zigbee, IOT, NB-IOT, and proprietary link protocols. Depending upon the implementation, the communication link may employ various communication circuitries 240, operating in one or more modes of transmission and/or receiving. For example, the communication circuitry 240 may include a wireless transceiver, a wireless transmitter, a wired transceiver, and a wired transmitter. The function of the communication link is to transmit and receive data to and from the wearable health monitoring device 200 to a cloud server 400. Depending on the implementation, the communication link may also be coupled to several wearable health monitoring devices to provide a network of sensors all connected to the receiving station 500.

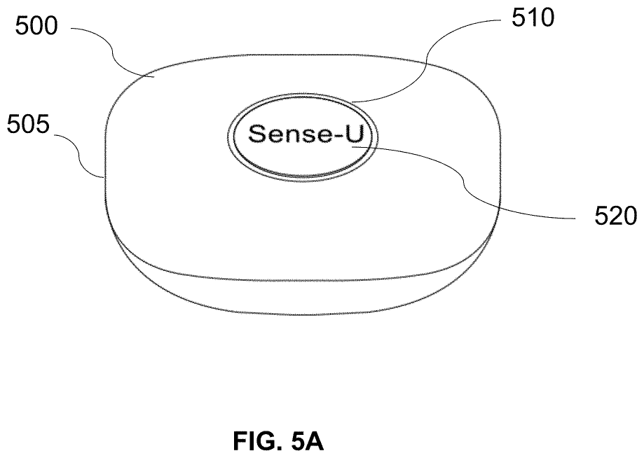

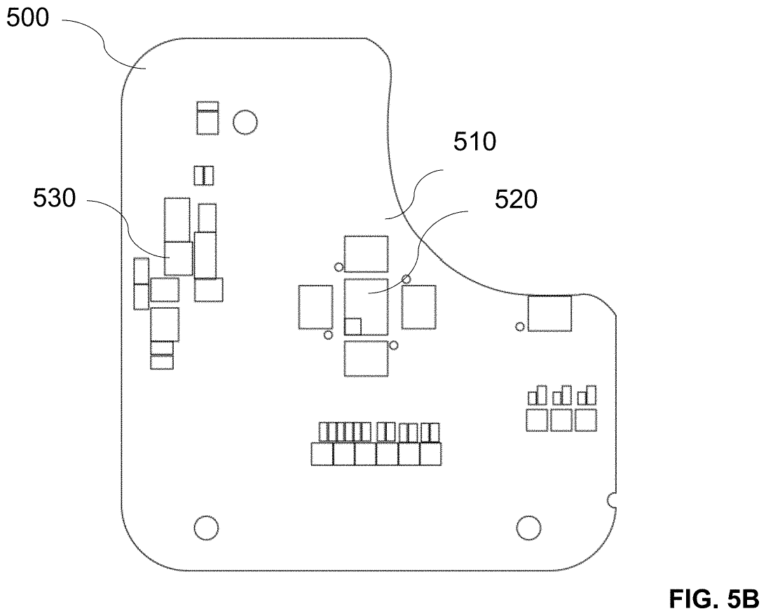

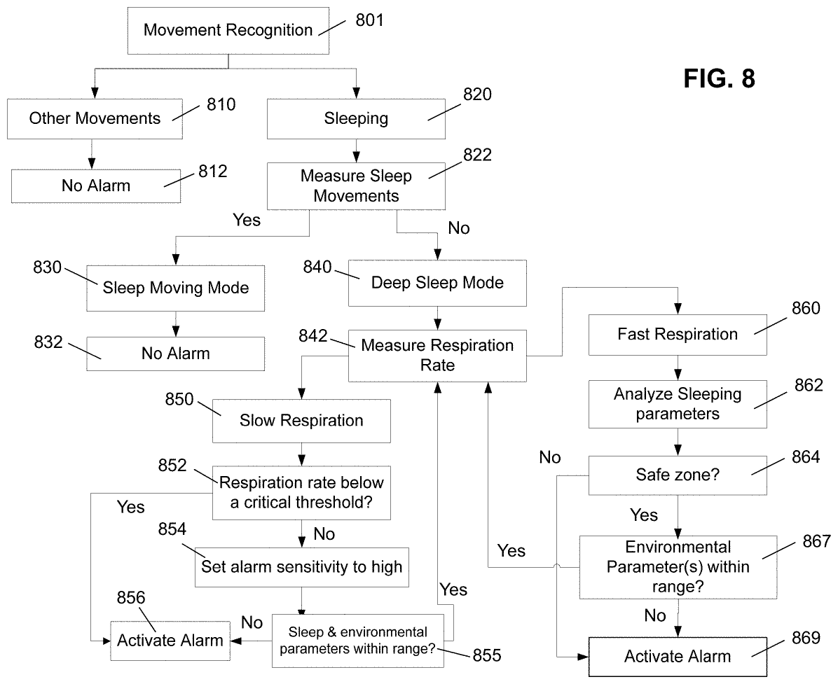

In some embodiments, referring to FIGS. 5A and 5B, a receiving station 500 includes an enclosure 505 with a light-emitting display 510, and a home button 520. The enclosure 505 can be made of a plastic material. The light-emitting display 510 can be implemented by a LED, e.g., a ring-shaped LED. The light-emitting display 510 allows parents or a guardian to check the health status of a baby's health easily and receive important health notifications. The home button 520 may make it easy for parents or a guardian to interrupt a health notification when something happens to their infants. In operation, the receiving station 500 is in communication with the wearable health monitoring device 200, the cloud server 400, and the mobile device 300. A computer processor 530 can analyze one or more signals or data from the movement sensors 210, the bio-vital signal sensors 220, and the environmental sensors 230 (as described below in relation to FIG. 8).

The receiving station 500 can receive the wireless transmission signal from a wearable health monitoring device 200, or from multiple wearable health monitoring devices 200 attached to different infants. The receiving station 500 can display the health indication data through the light-emitting display 510. The home button 520 on the enclosure of the receiving station may be of the type shown in the drawing and the picture.

Once the data has been processed and transmitted, the receiving station 500 can further process the data. In particular, the receiving station 500 can process the data and detect an abnormal trend in the received movement parameters (shown in FIG. 9B), e.g., respiration data (e.g., slow or fast respiratory rate), sleeping position data (e.g., stomach sleeping), or abnormal trend in the received environmental parameters (shown in FIG. 9C), e.g., low temperature, high temperature, temperature variations (temperature drop and temperature increment), high humidity level, or if the receiving station 500 detects a problem within the system (e.g., low battery, poor signal strength, or constant parameters for a certain long time period which indicates the sensor is out of its position, for example, the sensor is mis-positioning, or even fallen off from its originally clamped position, etc.) the receiving station 500 can provide an indication of the problem. For example, the receiving station 500 can sound an alarm, display a notification via the light-emitting display 510 of the receiving station 500, or otherwise send a message.

In some embodiments, referring to FIGS. 1 and 4, after receiving the data, the receiving station 500 can transmit the data to an internet gateway 110, such as a Wi-Fi router over the Internet to a cloud server 400, and further to the mobile device 300. Alternatively, the data can also be forwarded within a Wi-Fi network to the mobile device 300 directly without being required to transmit over the internet.

In some embodiments, the data can be transmitted to a mobile device 300 directly. In the case if the mobile device 300 detects an abnormal trend, the mobile device 300 can provide an indication of the problem. For example, the mobile device 300 can sound an alarm, display a notification on the screen of the mobile device 300, or otherwise send a message.

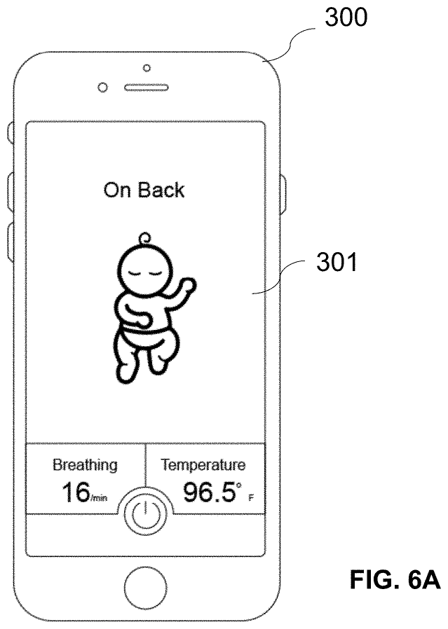

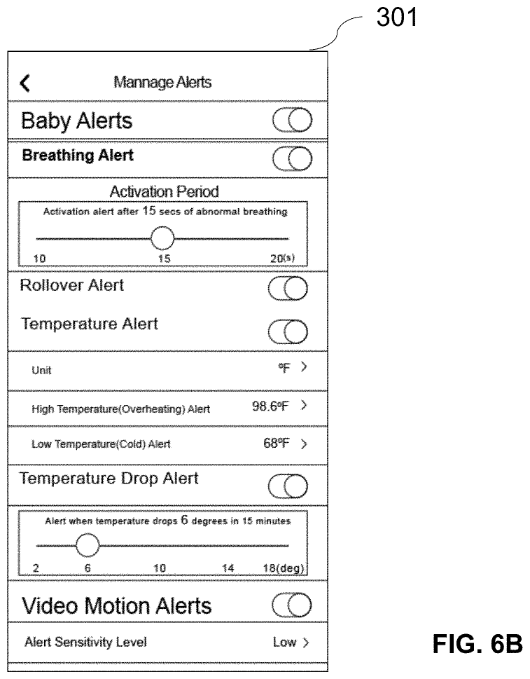

In some embodiments, the mobile device 300 can display analyses of the received movement data, bio-vital signal data, and the environmental data. For example, the mobile device 300 can include a user interface 301 that displays real-time respiratory rate, sleeping position and temperature of the infant (shown in FIG. 6A). Similarly, the mobile device 300 can show a graph tracking the respiratory rate, or temperature of an infant over time. Additionally, the user interface 301 can customize the settings of wearable health monitoring device and the alert threshold (shown in FIG. 6B). In general, the mobile device 300 can utilize the received information to display a variety of health data. In this way, when an abnormal health reading happens, a parent can receive an alert notification and further determine the urgency of the notification. The mobile device 300 can be in the form of a smart phone, a tablet computer, a personal computer, a laptop, or a customized computing device.

In some embodiments, a user can configure the receiving station 500's response to a particular alarm or to alarms in general. For example, a user can silence all alerts by tapping the ON/OFF button on the home screen of the application (FIG. 6A). Similarly, a user can also configure the receiving station 500 to only indicate an alarm if the alarm is enabled (FIG. 6B). Further, a user can configure the receiving station 500 to only indicate an alarm if certain conditions are met, e.g., temperature falling out of the range (FIG. 6B). Similarly, upon receiving an alert generated by the wearable health monitoring device 200, or upon receiving health data that demonstrates a negative trend, the mobile device 300 can also be configured to indicate an alert. For example, the mobile device 300 can sound an audible alarm, vibrate, or generate a visual alert. It should be understood that the presently disclosed systems and methods are compatible with a multitude of methods for receiving station 500 and the mobile device 300 to alert a user.

In addition, false alarms cause anxiety and unnecessary fear but many babies naturally hold their breath for short periods of time, causing slow and fast respiratory rates. Since this can be a normal occurrence, the base station 500 can include a delayed alarm mechanism. The mobile device 300 can adjust the activation period (as shown in FIG. 6B) to reduce or even avoid these false alarms.

In some embodiments, the high temperature threshold and the temperature increment in a predefined period can be used to monitor the sign of baby overheating and the low temperature threshold and the temperature drop parameters can be used to monitor the sign of getting cold.

The wearable health monitoring device 200 can integrate with a combination of movement sensors 210 as shown in FIG. 4A, and a combination of the environmental sensors 230 as shown in FIG. 4B. In this implementation, the mobile device 300 can alert a user to information of interest, including abnormal health trends.

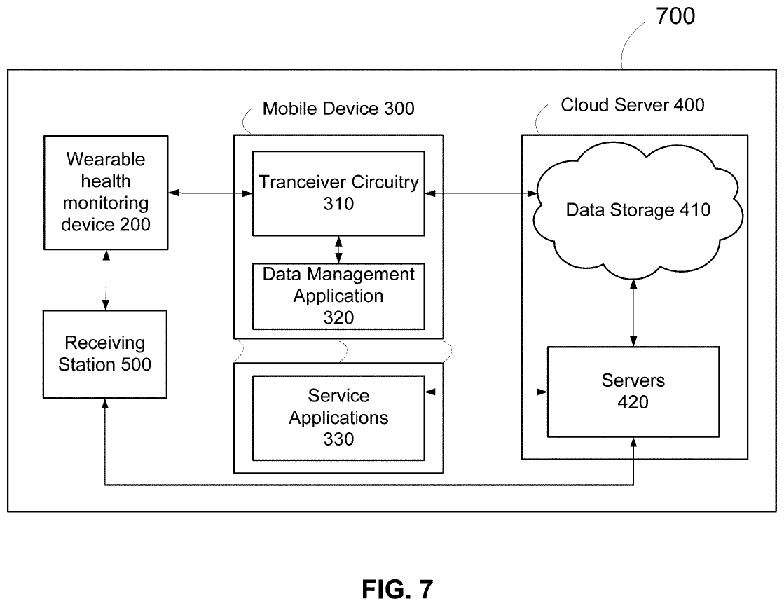

In some embodiments, referring to FIG. 7, a health monitoring system 700 includes a wearable health monitoring device 200, a mobile device 300, and a cloud server 400 comprising a data storage 410 and servers 420. The mobile device 300 can include a transceiver circuit 310, data management applications 320, and service applications 330. The cloud server 400 can access a historical record of health recordings, and video clips. For example, a parent or a guardian of an infant can access a historical record of the infant's breathing and related health data and provide the record to the infant's doctor. The remote cloud server 400 can access a historical record of health recordings. For example, a parent of an infant can access a historical record of the infant's breathing and related health data and provide the record to the infant's doctor. The accessed historical record can be stored by the cloud server 400, the receiving station 500, the mobile device 300, or some other web-based storage cache. Optionally, the health monitoring system 700 can include a receiving station 500. As disclosed above in FIGS. 5A and 5B, the wearable health monitoring device 200 can communicate with the receiving station 500, which can in turn communicate with the mobile device 300 and/or the cloud server 400.

The mobile device 300 can receive the wireless signal from the wearable health monitoring device 200 directly, or from the server 400. The mobile device 300, as shown in FIGS. 6A and 6B above, can then display the data on its screen, including real-time breathing data, visualized sleeping positions, temperature, etc. It can also distinguish between the potentially multiple wearable health monitoring devices 200.

In some embodiments, a health monitoring system can include the wearable health monitoring device 200, the receiving station 500, the mobile device 300, and the cloud server 400. In this implementation, the wearable health monitoring device 200 can communicate with the receiving station 500, which can in turn communicate with the cloud server 400, and/or the mobile device 300. Both the receiving station 500 and the mobile device 300 can alert a user to information of interest, including abnormal health trends.

Furthermore, in some embodiments, a health monitoring system can include the wearable article 100, the wearable health monitoring device 200, the receiving station 500, the mobile device 300, and the cloud server 400. In this implementation, the wearable article 100 integrated with the wearable health monitoring device 200, forms a smart diaper and can communicate to the receiving station 500 through a wireless protocol. The receiving station 500 can then transmit information to a remote cloud server 400 of interest.

An exemplified process associated with the presently disclosed health monitoring system is now described. First, the disclosed health monitoring system monitors the proper attachment of the wearable health monitoring device to the wearer, such as an infant. If it is found that the wearable health monitoring device is not properly attached or disposed in a wearable article (such as a diaper or a swaddle blanket), the health monitoring system sends a warning that allows a parent, a care taker, or the wearing person to adjust the placement of the wearable health monitoring device.

Referring to FIGS. 8 and 9A, using the monitoring the health of an infant as an example, the movement pattern of an infant wearing the wearable health monitor device is recognized (step 801) based on the movement data detected by one or more movement sensors in the wearable health monitoring device 200. Examples of movement patterns 210A (FIG. 9A) that can be recognized by the disclosed health monitoring system can include sleeping 210A.1, moving 210A.2, sitting 210A.3, standing 210A.4, walking 210A.5, running 210A.6, and the wearable health monitoring device detached 210A.7.

If it is determined that the infant is not sleeping 210A.1 but in one of other movement patterns such as moving 210A.2, sitting 210A.3, standing 210A.4, walking 210A.5, running 210A.6, or detached modes 210A.7 (step 810), no alarm will be activated (step 812) related to the prevention of Sudden Infant Death Syndrome. It should also be noted that movement data can be used to analyze and produce alarms for other movement behaviors. For example, when the movement data show that a baby is climbing the guardrail of, or fallen from a babe bed, an alarm signal will be sent to the parent or guardian.

If it is determined that the infant is sleeping 210A.1 (step 820), sleep movements are measured (step 822). If the intensity of movements during sleep is over a threshold, the infant will be considered to be in a sleep moving mode (e.g., waggling, which can be considered as an instance of the moving mode 210A.2) (step 830). The infant is determined to be in a safe state and no alarm will be produced (step 832).

If the amount of movements is below a threshold, the infant will be considered to be in a deep sleep mode (step 840), the disclosed health monitoring system measures one or more bio-vital signals such as the respiratory rate (step 842) using one or more of the bio-vital signal sensor (220 in FIG. 4B).

If the measured respiratory rate is below a respiration threshold, a person, e.g., an infant is determined to be in a slow respiration mode (step 850). The disclosed health monitoring system checks if the respiratory rate is below a critical threshold (step 852). If it is, which includes the situation of a stop of breathing for a certain period of time that is longer than the preset threshold period and an alarm is produced (step 856). Moreover, a threshold amount of time can be allowed for the health readings to return to a normal level. If the respiratory rate is above a critical threshold but below lower normal threshold, the disclosed health monitoring system sets the alarm sensitivity level to high (step 854). Furthermore, it checks if one or more of the sleeping parameters (210B) and the environmental parameters (220A) are within range (step 855), e.g. sleeping position (210B.2 in FIG. 9B) of the infant using the movement data from one or the movement sensors such as an accelerometer (211, FIG. 4A) disposed next to the stomach of the infant. If a detected sleeping/environmental parameter is out of the predefined range, e.g. sleeping on stomach, then the disclosed health monitoring system can conclude that the abnormal respiratory rate may be related to the specific sleeping/environmental parameter(s). Therefore, the disclosed health monitoring system produces an alarm signal indicating that the slow respiratory rate alarm which might be related to the specific sleeping/environmental parameter(s) (step 856). The alarm signal can be in one or a combination of forms such as audible, visual, vibration, or an electronic text, etc. If it is detected that the infant is sleeping in a safe posture (e.g., on the back), the health monitoring system returns to measuring and continuing to monitor respiratory rate (842).

Exemplified sleeping parameters are shown in FIG. 9B. The calculated movement parameters 210B include, but are not limited to standing/sitting posture 210B.1, sleeping position 210B.2, rollover 210B.3, breathing movement 210B.4, breathing/respiratory rate 210B.5, heart rate/pulse rate 210B.6, snoring 210B.7, steps/di stance/calories 210B.8, and sleep quality 210B.9.

If the measured respiratory rate (in step 842) is above a respiration threshold, the infant will be considered to be in a fast respiration mode (step 860). The health monitoring system analyzes the corresponding sleeping parameters (FIG. 9B) (step 862). If the period of time that the infant stays at such fast respiration is for an extended period longer than a threshold period, it is then determined whether the respiration behaviors are within an intermittent zone (step 864). If the respiration behaviors do not meet criteria for an intermittent zone (e.g. respiration is overly fast or for a longer enough period of time) (step 864), the health monitoring system activates alarm (step 869).

Next, if the respiration behaviors meet criteria for an intermittent zone, the health monitoring system can further set the alarm sensitivity level to high and measure one or more environmental parameter and determine whether an environmental parameter measured is out of range (step 867). Examples of environmental parameters 220A (FIG. 9C) can include, but are not limited to body/skin temperature 220A.1, ambient temperature 220A.2, temperature change in a predefined period 220A.3, humidity level 220A.4 (that can include measurement data on ambient humidity and the humidity in a wearable article such as a diaper), ultra-violet intensity level 220A.5, audio noise level, electro-magnetic wave strength level, and location/position 220A.6. If one or more environmental parameters are out of safe range (e.g. the ambient temperature, the ambient humidity, or surrounding audio noise level over respective desirable ranges), the health monitoring system produces an alarm (step 869) to alert the parents or guardians to check on health status, e.g., the fast breathing status. If one or more environmental parameters are within safe range (e.g. the ambient temperature or the ambient humidity within respective desirable ranges), the health monitoring system returns to measuring and monitoring respiratory rate (step 842).

Throughout the process, the health monitoring system continues monitor movement patterns (step 801). If the infant wakes up and exhibit movement behaviors other than sleep (step 810), the health monitoring system considers the infant to be in a safe state (step 812).

It should be noted that although part of the above process is described using an infant as an example, the disclosed process and system are applicable to persons of other ages. The described operation steps are consistent with persons of older age wearing the disclosed wearable health monitor device. An example for a need for such system is when a child has a fever or pneumonia risk, wherein high body temperature and fast respiratory rate are common symptoms, and the monitoring of respiratory rate and body temperature can be valuable for early intervention. Another example for a need for the disclosed health monitoring system is someone having sleep apnea in which breathing repeatedly stops and starts, wherein the monitoring of respiratory rate and early intervention can be valuable. The third example for a need for such system is an elderly person who has Alzheimer's disease. The alarm signals can be sent to his or her guardian or caretaker. The disclosed health care system can be used to detect early symptoms of Alzheimer's disease and Parkinson's disease in a person wearing the disclosed wearable heath monitoring device. The disclosed health care system can also be used to help patients to delay, slow down, or prevent the development of Alzheimer's disease and Parkinson's disease. The disclosed health care system can also be used to monitor, and/or prevent, and alert respiration issues of elderly persons. Another example for a need for the disclosed health monitoring system is someone having asthma, wherein the monitoring of respiratory rate and blood pulse can be valuable for early intervention.

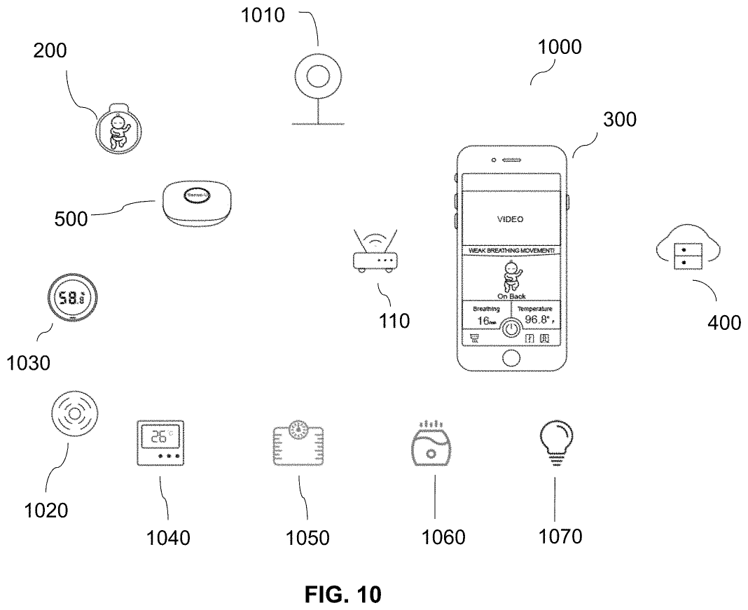

The above described operation steps can be implemented by one or multiple devices including the wearable health monitoring device 200 (FIGS. 1, 4, 7, and 10), the mobile device 300 (FIGS. 1, 6A, 6B, 7, 10), the receiving station 500 (FIGS. 5A, 5B, 10), the remote cloud server 400 (FIGS. 1, 7, and 10), or other devices compatible with the presently disclosed system. For example, the analyses and the recognition of the wearing person's movement can be conducted on the wearable health monitoring device 200 (FIGS. 1, 4, 7, and 10), while further analyses can be conducted on the mobile device 300 (FIGS. 1, 6A, 6B, 7, 10), the receiving station 500 (FIGS. 5A, 5B, 10), or the remote cloud server 400 (FIGS. 1, 7, and 10). In some embodiments, all the analysis steps in FIG. 8 can be conducted on the mobile device 300 (FIGS. 1, 6A, 6B, 7, 10), or the receiving station 500 (FIGS. 5A, 5B, 10), or the remote cloud server 400 (FIGS. 1, 7, and 10).

In general, an abnormal reading can consist of abnormal respiratory rate reading that falls out of a predefined range. Additionally, abnormal readings can also represent a temperature reading or temperature variation that falls out of a predefined range. Further, abnormal readings can also consist of a stomach sleeping position or an unhealthy breathing movement waveform. It should be understood that the described abnormal readings are not meant as an exhaustive list of the abnormalities that the presently disclosed systems and methods can identify and compensate for.

It should be noted that the above disclosed operation steps can be implemented using machine learning. A deep learning model can be trained by movement data, bio-vital signal data, and environmental parameters data and known conditions of the wearing person. The trained model can be used separately or in combination with the flowchart disclosed above to automatically determine the state of the wearing person and the need for generating an alarm.

FIG. 10 illustrates an infant monitoring system 1000 for monitoring the health of an infant. In particular, a mobile device 300 is in communication with a variety of infant monitoring devices, for example, a wearable health monitoring device 200, a video monitor 1010, a detector 1020 for smoke and/or carbon monoxide, and a room thermometer 1030. In some embodiments, an infant monitoring system can include one or more of these devices in combination. For example, the wearable health monitoring device 200 may detect an abnormal health reading. In response to the reading, the mobile device 300 can request a video clip transmitted from a video monitor 700, smoke and carbon monoxide readings from a detector 1020, or temperature/humidity readings from a room thermometer 1030. The video monitor 1010 can generate detected movements of the wearing person and its output signals can be sued to generate video motion alerts (shown in FIG. 6B). Moreover, the video monitor 1010 can also monitor sound including breathing sound produced by the infant. The health monitoring system can analyze breathing noise and detect signs of breathing difficulties and choking risks such as blocking of air channels by mucus, or by covering of mouth by objects such as toys or pillows, or head's face-down positions. In this way, a parent can receive a mobile notification that an abnormal health reading has occurred, while at the same time receiving more data of the infant to determine whether the situation is an emergency.

In some embodiments, a video monitor 1010 can capture videos of the infant and streams them to the smart device 300 via a router device 110. When an abnormal health reading happens, a parent can receive an alert notification, together with a corresponding video clip, to further determine the urgency of the notification.

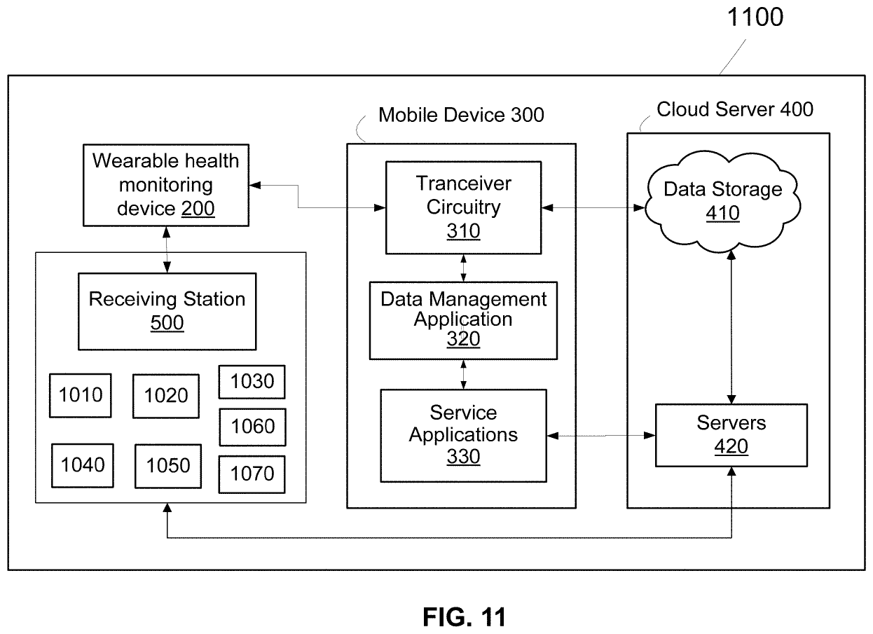

In some embodiments, referring to FIG. 11, a health monitoring system 1100 can include a wearable health monitoring device 200, a mobile device 300, a cloud server 400, an optional receiving station 500, a video monitor 1010, a detector 1020 for smoke and/or carbon monoxide, and a room thermometer 1030. The health monitoring system 1100 can also include a smart thermostat 1040 for ambient temperature control, a smart scale 1050 that can measure body weights of an infant, and a smart humidifier and dehumidifier 1060 that can automatically adjust ambient humidity level based on the measured ambient humidity. The receiving station 500, the video monitor 1010, the detector 1020, the room thermometer 1030, the smart thermostat 1040, the smart scale 1050, and the smart humidifier and dehumidifier 1060 can transmit the data to an internet gateway 110 such as a Wi-Fi router over the Internet to the cloud server 400, and further to the mobile device 300.

The cloud server 400 includes data storage 410 and servers 420. The mobile device 300 can include a transceiver circuit 310, data management applications 320, and service applications 330. The wearable health monitoring device 200 can be in communication with a receiving station 500. The receiving station 500 can be in communication with an internet gateway 110 (e.g., a cable modem, a Wi-Fi router, a DSL modem, etc.). In one application, the health monitoring system 1100 can be applied for infant care to monitor medical events such as apnea.

The cloud server 400 can access a historical record of health recordings, and video clips. For example, a parent can access historical record of the infant's breathing and related health data and provide the record to the infant's doctor. The historical record can be stored by the cloud server 400, the receiving station 500, the mobile device 300, or some other web-based storage cache. The health monitoring system 1100 can include a receiving station 500. As disclosed above in FIGS. 5A and 5B, the wearable health monitoring device 200 can communicate with the receiving station 500, which can in turn communicate with the mobile device 300 and/or the cloud server 400. Both the receiving station 500 and the mobile device 300 can alert a user to information of interest, including abnormal health trends.

The mobile device 300 can receive the wireless signal from the wearable health monitoring device 200 directly, or from the server 400. The mobile device 300, as shown in FIGS. 6A and 6B above, can then display the data on its screen, including real-time breathing data, visualized sleeping positions, temperature, historical records of health data, etc. It can also distinguish between the potentially multiple wearable health monitoring devices 200.

In some embodiments, a health monitoring system can include the wearable article 100 (e.g. FIG. 2A) that can be integrated with the wearable health monitoring device 200, forms a smart diaper and can communicate to the receiving station 500 through a wireless protocol. The receiving station 500 can then transmit information to a remote cloud server 400 of interest. An exemplified process associated with the presently disclosed health monitoring system is now described. First, the disclosed health monitoring system monitors the proper attachment of the wearable health monitoring device to the wearer, such as an infant. If it is found that the wearable health monitoring device is not properly attached or disposed in a wearable article (such as a diaper or a swaddle blanket), the health monitoring system sends a warning to allow a parent, a care taker, or the wearing person to adjust the position of the wearable health monitoring device.

The health monitoring system 1100 can monitor the health of an infant. In particular, a mobile device 300 is in communication with the wearable health monitoring device 200, the video monitor 1010, the detector 1020 for smoke and/or carbon monoxide, and the room thermometer 1030. The health monitoring system 1100 can include one or more of these devices in combination. For example, the wearable health monitoring device 200 may detect reading of an abnormal bio-vital signal. In response to the reading, the mobile device 300 can request a video clip transmitted from a video monitor 700, smoke and carbon monoxide readings from the detector 1020, or temperature/humidity readings from a room thermometer 1030. The video monitor 1010 can record detected movements of the wearer and generate video motion alerts (shown in FIG. 6B). In this way, a parent can receive a notification that an abnormal health reading has occurred, while at the same time receiving more data of the infant to determine whether the notification is an emergency.

In some embodiments, a video monitor 1010 can capture videos of the infant and streams them to the smart device 300 via a router device 110. When an abnormal health reading happens, a parent can receive an alert notification, together with a corresponding video clip, to further determine the urgency of the notification.

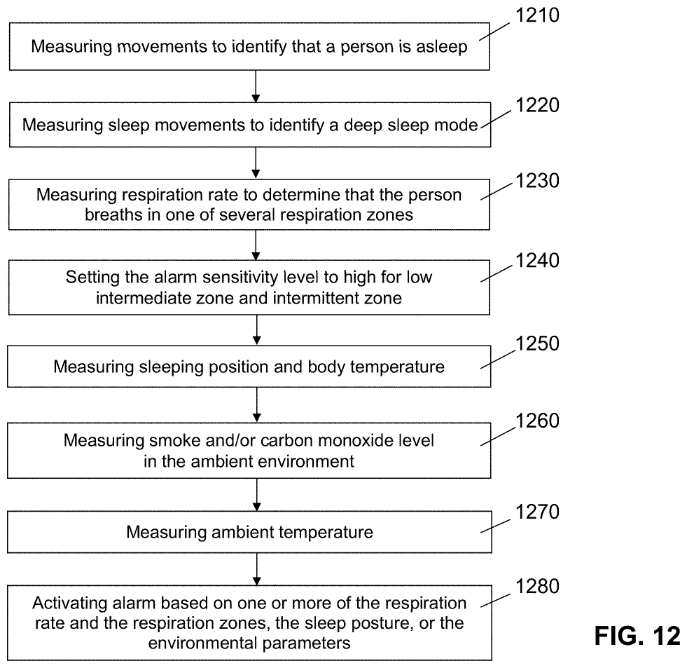

Referring to FIGS. 9A, 11, and 12, using infant monitoring as an example, the movement patterns of an infant wearing the wearable health monitor device are recognized based on the movement data detected by one or more movement sensors in the wearable health monitoring device 200 (step 1210) (FIG. 8). Examples of movement patterns 210A (FIG. 9A) that can be recognized by the disclosed health monitoring system can include sleeping 210A.1, moving 210A.2, sitting 210A.3, standing 210A.4, walking 210A.5, running 210A.6, and the wearable health monitoring device detached 210A.7.

If it is determined that the infant is not sleeping 210A.1 but in one of other movement patterns such as moving 210A.2, sitting 210A.3, standing 210A.4, walking 210A.5, running 210A.6, or detached modes 210A.7, no alarm will be activated related to the prevention of Sudden Infant Death Syndrome (step 1210). It should also be noted that movement data can be used to analyze and produce alarms for other movement behaviors. For example, when the movement data show that a baby is climbing the guardrail of, or falling from a crib, an alarm signal will be sent to the baby's guardian.

If it is determined that the infant is in a sleep mode (FIG. 8), sleep movements are measured (step 1210). If the amount of movements during sleep is over a threshold, the infant will be considered to be in a sleep moving mode (which can be considered as an instance of the moving mode 210A.2). The infant is determined to be in a safe state and no alarm will be produced (FIG. 8).

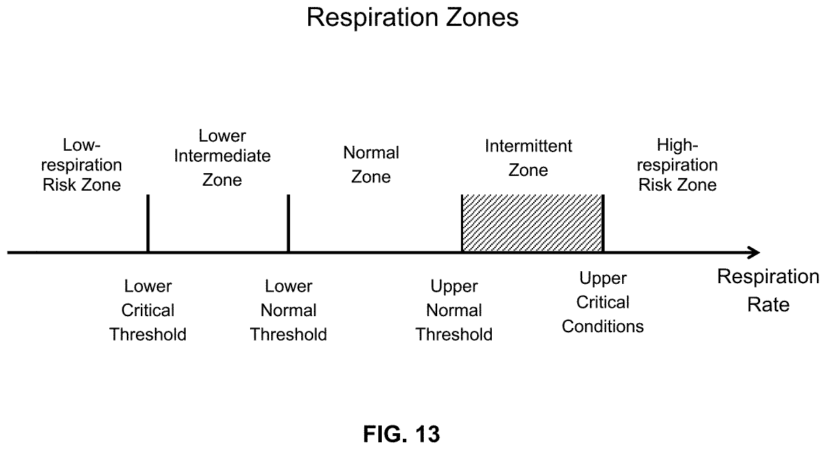

If the amount of movements is below a threshold, the infant will be considered to be in a deep sleep mode (step 1220), the disclosed health monitoring system conducts a measurement of one or more bio-vital signals such as the respiratory rate of the infant (step 1230) using the wearable health monitoring device 200 (which include one or more bio-vital signal sensors 220 in FIG. 4B) to identify the respiration zone (FIG. 13) that the infant is in.

If the measured respiratory rate is below a respiration threshold, the infant is determined to be in a slow respiration mode (step 1230). The disclosed health monitoring system checks if the respiratory rate is below a lower critical threshold for a period longer than a predetermined period of time (FIG. 13), which includes the situation of a stop of breathing. If it is, the person is a low-respiration risk zone. An alarm is produced (step 1280) immediately.

If the respiratory rate is above the lower critical threshold but below a lower normal threshold (FIG. 13), the infant is breathing in a lower intermediate zone. The disclosed health monitoring system automatically sets the alarm sensitivity level to high (step 1240). and checks if one or more of the sleeping parameters (210B) and the environmental parameters (220A) is within range step (1250). For example, the sleeping position (210B.2 in FIG. 9B) of the infant can be determined using the wearable health monitoring device 200 (including for example accelerometer sensor 211 in FIG. 4A) disposed next to the stomach of the infant. In some embodiments, the sleeping position (210B.2 in FIG. 9B) of the infant can also be obtained in combination with the video camera 1010. If the detected sleeping position is on stomach, the respiratory rate is determined to be abnormal for the stomach sleeping. Therefore, the disclosed health monitoring system produces an alarm signal indicating that the slow respiratory rate alarm which might be related to the stomach sleeping (step 1280). The alarm signal can be in one or a combination of forms such as audible, visual, vibration, or an electronic text, etc. If it is detected that the infant is sleeping in a safe posture (on the back or a side), the health monitoring system returns to measuring and continuing to monitor respiratory rate (step 1230).

If the measured respiratory rate is between the lower normal threshold and an upper normal threshold (FIG. 13), the infant breathing is considered to be in a normal zone by the health monitoring system (step 1230). No breathing alarm will be produced.

If the measured respiratory rate is above the upper normal threshold (FIG. 13), the infant will be considered to be in a fast respiration mode (step 1230). The health monitoring system analyzes the absolute values of the fast respiratory rate and the period of time that the infant stays at such fast respirations. If the respiration data meets an upper critical condition (staying at above the upper normal threshold for long than a predetermined period) (FIG. 13), the person is in a high-respiration risk zone. The health monitoring system activates alarm (step 1280). If the respiration data does not meet the upper critical condition (does not stay at above the upper normal threshold for long than a predetermined period) (FIG. 13), the health monitoring system determines that the respiration behaviors are within an intermittent zone (FIG. 13). No breathing alarm is produced.

The healthy respirations of young children are dependent on their ages: for a newborn or infant (0-12 months), the normal respiratory rate is 30-60 breaths per minutes; for a toddler (1-3 years), the normal respiratory rate is 24-40 breaths per minutes; These exemplified normal ranges define in the lower normal threshold and the upper normal threshold for the respiratory rates at different ages. When a newborn's respiratory rate is below a predefined lower critical respiration threshold (e.g. 10 breaths per minutes), a slow breathing alarm will sound. When a newborn' respiratory rate is higher than a predefined higher critical respiration threshold (e.g. 60 breaths per minutes), a fast breathing alarm will be produced.

In some embodiments, the threshold respiratory rates of the respiration zones can become more protective if the infant or baby is having a fever or pneumonia. For example, the lower critical threshold may be raised under such conditions to create a safety margin. In the intermittent zone, the period of time that the infant is allowed to have respiratory rate staying above the upper normal threshold may also be shortened to be more protective.

If the respiration behaviors are in a normal zone or the intermittent zone, the wearable health monitoring device 200 also measures the body temperature of the infant (step 1250). If the infant body temperature is out of a normal range (above or below the range), the health monitoring system activates alarm (step 1280). In some embodiments, smart thermostat can set the room temperature to preset ranges and can automatically adjust the ambient temperature when the infant's body temperature or the measured ambient temperature deviates from preset values.

The health monitoring system can further measure one or more environmental parameter and determines whether an environmental parameter measured is out of range. Examples of environmental parameters 220A (FIG. 9C) can include, but are not limited to body/skin temperature 220A.1, ambient temperature 220A.2, temperature change in a predefined period 220A.3, humidity level 220A.4 (that can include measurement data on ambient humidity level and the humidity level in a wearable article such as a diaper), ultra-violet intensity level 220A.5, audio noise level, electro-magnetic wave strength level, and location/position 220A.6. In particular, the detector 1020 can continuously monitor smoke including secondhand smoke and carbon monoxide level in the ambient air (step 1260). In some implements, a smart air purifier can automatically capture carbon monoxide and PM 2.5 pollutants such as smoke, allergens, odors, mold spores, dust mites and pet dander, etc., based on the measured carbon monoxide and smoke level by the detector 1020, and/or the respiratory rate measured by the wearable health monitoring device 200.

The room thermometer 1030 can continuously measure ambient temperatures in the ambient air (step 1270). In some embodiments, thermometer is integrated with or inside the receiving station 500. If one or more environmental parameters are out of preset range (e.g. the ambient temperature or the ambient humidity level over respective desirable ranges), the health monitoring system produces an alarm (step 1280) to alert the parents to check on the environment of the infant. In some embodiments, a room thermostat 1040 and a smart humidifier and dehumidifier 1060 can automatically adjust ambient temperature and humidity level based on the measured ambient humidity level as described above.

In some embodiments, an alarm signal is produced if the video monitor 1010 detects that the infant's face is covered by an object or co-sleeping with others.

If no alarm is produced during the above steps, the health monitoring system returns to measuring movement data (step 1210), monitoring sleep mode (sleep 1220), and monitoring respiratory rate (step 1230).

In some embodiments, the health monitoring system 1100 can further include other monitoring devices. A smart scale 1050 can regularly measure body weights of the infant and send the measured weights to the mobile device 300 and the cloud server 400, to draw baby growth charts automatically.

Moreover, a smart light bulb 1070 can automatically adjust ambient luminescence based on the data collected from other devices, for example, dimming/turning off the light when the baby is determined to be asleep, and increasing the luminescence/turning on the light when the wearable health monitoring device 200 detects that the infant is awake.

In addition, the bio-vital signals measured by the wearable health monitoring device 200 can include heartbeat and blood oxygen levels, which are communicated to the mobile device 300. Alerts are generated when heartbeat is abnormal or blood oxygen level is low.

Ambient sounds and noises can be measured by a microphone or as part of measurements by the video monitor 1010.

Accordingly, FIGS. 1-12 provide a number of components, schematics, and mechanisms for wirelessly monitoring the health of an infant. In particular, a wireless wearable health monitoring device 200 communicates health data from an infant to a receiving station 500. The receiving station 500 can then be used to monitor the infant's health or to alert an individual to a negative trend in the infant's health. Additionally, false alarms can be detected, and in some cases prevented, by analyzing trends in the received health data.

The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for monitoring health of a person by a health monitoring system, comprising:
measuring movement data of a person using a wearable health monitoring device;
determined whether the person is in a deep sleep mode based on the movement data;
automatically measuring a respiratory rate of the person that is in a deep sleep mode;
determining that the person is in one of a plurality of respiration zones;
measuring at least one of a body temperature, an ambient temperature, a sleeping position, a smoke or carbon monoxide level, or images of the person; and
automatically producing an alarm signal if a predetermined criterion is met based on the respiration zone and at least one of the body temperature, the ambient temperature, a sleeping position, the smoke level, the carbon monoxide level, or the images of the person.

2. The method of claim 1, wherein the plurality of respiration zones includes a normal zone bounded by a lower normal threshold and an upper normal threshold, wherein the plurality of respiration zones includes a low-respiration risk zone defined by a lower critical threshold below the lower normal threshold, wherein the alarm is automatically produced if the respiratory rate is below the lower critical threshold.

3. The method of claim 2, wherein the plurality of respiration zones further includes a lower intermediate zone between the low-respiration risk zone and the normal zone,
wherein an alarm sensitivity level is set to a highest level if the person is determined in the lower intermediate zone.

4. The method of claim 3, wherein the alarm is produced if the person is determined to be sleeping on stomach or a side based on the sleeping position measurement from the wearable health monitoring device or the video monitor.

5. The method of claim 1, wherein the plurality of respiration zones further includes an intermittent zone and a high-respiration risk zone having respiratory rates above the upper normal threshold, wherein the person is determined to be in the intermittent zone with no alarm being produced if the respiratory rate is above the upper normal threshold but for less than a predetermined period of time, the alarm sensitivity level is set to the highest level if the person is determined in the intermittent zone.

6. The method of claim 5, wherein the person is determined to be in the high-respiration risk zone and the alarm is produced if the respiratory rate is above the upper normal threshold but for longer than the predetermined period of time.

7. The method of claim 1, wherein the alarm is produced if the body temperature of the person is out of a pre-specified range.

8. The method of claim 1, further comprising:
automatically measuring an ambient temperature or ambient humidity,
wherein the alarm signal is produced if the ambient temperature or the ambient humidity is determined to be outside of a preset range.

9. The method of claim 1, further comprising:
automatically adjusting the levels of ambient temperature or the ambient humidity if measured ambient temperature or measured ambient humidity is respectively out of a preset range.

10. The method of claim 1, wherein the alarm signal is produced if the smoke level or the carbon monoxide level is outside a predetermined safe range.

11. The method of claim 1, further comprising:
measuring the person's body weight and automatically updating the growth chart of a person.

12. The method of claim 1, wherein the person is an infant, baby, toddler, teenage, or adult.

13. The method of claim 12, wherein the wearable health monitoring device is attached to or removably disposed in a wearable article worn adjacent to the baby's abdomen.

14. A health monitoring system, comprising:
a wearable health monitoring device comprising one or more movement sensors configured to produce movement data of a person that wears the wearable health monitoring device; and
one or more computer processors configured to determine whether the person is in a deep sleep mode based on the movement data,
wherein when the person is determined to be in the deep sleep mode, the wearable health monitoring device is configured to measure a respiratory rate of the person,
wherein the one or more computer processors are configured to determine that the person is in one of a plurality of respiration zones,
wherein an alarm signal is produced if a predetermined criterion is met based on the respiration zone and at least one of a body temperature, an ambient temperature, a sleeping position, a smoke or carbon monoxide level, or images of the person.

15. The health monitoring system of claim 14, wherein the wearable health monitoring device is configured to measure the respiratory rate and the body temperature of the person, wherein the alarm is produced if the body temperature of the person is out of pre-specified range.

16. The health monitoring system of claim 14, further comprising:
a video monitor configured to record the images of the person;
a detector configured to measure the smoke and carbon monoxide levels in ambient environment;
a room thermometer configured to measure the ambient temperature and humidity level; and
a smart scale configured to measure the weight of the person.

17. The health monitoring system of claim 14, wherein the plurality of respiration zones includes a normal zone bounded by a lower normal threshold and an upper normal threshold, wherein the plurality of respiration zones includes a low-respiration risk zone defined by a lower critical threshold below the lower normal threshold, wherein the alarm is automatically produced if the respiratory rate is below the lower critical threshold.

18. The health monitoring system of claim 17, wherein the plurality of respiration zones further includes an intermittent zone and a high-respiration risk zone having respiratory rates above the upper normal threshold, wherein the person is determined to be in the intermittent zone with no alarm being produced if the respiratory rate is above the upper normal threshold but for less than a predetermined period of time, and the alarm sensitivity level is set to the highest level if the person is determined in the intermediate zone.

19. The health monitoring system of claim 14, wherein the person is determined to be in the high-respiration risk zone and the alarm is produced if the respiratory rate is above the upper normal threshold but for longer than the predetermined period of time.

20. The health monitoring system of claim 14, wherein the wearable health monitoring device is attached to or removably disposed in a wearable article worn adjacent to the baby's abdomen.
